(19) United States
(12) Patent Application Publication
(10) Pub. No.: US 2021/0290719 A1
(43) Pub. Date: Sep. 23, 2021

(54) PLANT EXTRACT PRODUCT

(71) Applicant: Universal Village International Pty Ltd, Hawthorn (AU)

(72) Inventors: Nicole LaMond, Hawthorn (AU); Fred Hollamby-Jones, Hawthorn (AU)

(21) Appl. No.: 17/250,578

(22) PCT Filed: Aug. 1, 2019
(86) PCT No.: PCT/AU2019/050807

§ 371 Date: Feb. 4, 2021

(30) Foreign Application Priority Data
Aug. 4, 2018 (AU) 2018902844

(51) Int. Cl. A61K 36/752; A61K 36/48; A61K 36/53; A61K 36/185; A61K 36/61; A61K 9/00; A23L 33/105; A23P 10/20; A23F 3/14; A23F 3/32

(52) U.S. Cl. CPC A61K 36/752; A61K 36/48; A61K 36/53; A61K 36/185; A61K 36/61; A23V 2002/00; A23L 33/105; A23P 10/20; A23F 3/14; A23F 3/32; A61K 9/0095

(57) ABSTRACT

The application relates to a method of producing plant extract agglomerates suitable for a beverage product, the method comprising rewet agglomerating plant extract powder wherein all processing and drying is performed at low temperatures to substantially avoid decreasing the nutritional value of the plant extract powder. The resulting plant extract agglomerate can be used in beverage products such as tea, herbal teas and coffee so as to increase the nutritional value of the beverage product.

### CROSS-REFERENCE TO RELATED APPLICATION(S)

This application is a Section 371 National Stage Application of International Application No. PCT/AU2019/050807, filed Aug. 1, 2019 and published as WO 2020/028935 A1 on Feb. 13, 2020, in English, and further claims priority to Australian Provisional Patent Application Ser. No. 2018902844, filed Aug. 4, 2018, the contents of each of which are hereby incorporated by reference in their entirety.

### FIELD

The present invention is generally directed to beverage products and in particular to an agglomerated plant extract suitable for use in tea, herbal teas, coffee and other beverages.

### BACKGROUND OF THE INVENTION

It is well understood that vitamins are essential to good health. For example: vitamin A can regulate cell and tissue growth, vitamin D regulates mineral metabolism, vitamin E functions as an antioxidant. Insufficient intake of vitamins can cause clinically significant illness such as scurvy, rickets, and beriberi.

As vitamins and their effect on health have become better understood, concern has grown that modern diets contain insufficient vitamins. As a result the popularity of vitamin supplements has grown. However, despite their popularity, it is common for people to forget or neglect to take vitamin supplements, even after purchase. Since vitamin tablets are typically designed for function rather than taste or ease of ingestion, the taking of such tablets may not be enjoyable and can form part of a disdained daily dosage regimen rather than an enjoyable daily ritual.

At the same time as concerns over nutrient content in modern diets have arisen, concerns have also grown over the detrimental effect of artificial chemicals on human health. Organic foods, and organic products in general, have increased in popularity as people strive to prevent or minimise their intake of artificial chemicals and additives.

The above discussion of background art is included to explain the context of the present invention. It is not to be taken as an admission that the background art was known or part of the common general knowledge at the priority date of any of the claims of the specification.

It would therefore be advantageous to provide an alternative source of vitamins which is one of more of:
(a) high in a broad range of vitamins;
(b) naturally derived (i.e. containing minimal if any artificial additives); and
(c) can be simply and readily incorporated into modern diets through common food or beverage products.

### SUMMARY OF THE INVENTION

According to one aspect of the invention there is provided a method of producing plant extract agglomerates suitable for a beverage product, the method comprising rewet agglomerating plant extract powder wherein all processing and drying is performed at low temperatures to substantially avoid decreasing the nutritional value of the plant extract powder.

Preferably, the method is performed using fluidized bed rewet agglomeration.

Preferably, the processing and drying is performed at below 65° C.

Preferably, the method comprises wetting the plant extract powder with water.

Preferably, the plant extract powder comprises one or more plant extracts of: agati (Sesbania grandiflora); holy basil (Ocimum sanctum), lemon (Citrus limon), Indian gooseberry (Phyllanthus emblica), common guava (Psidium guahava), and achiote (Bixa orellana).

According to a further aspect of the invention there is provided plant extract agglomerates produced according to the above method.

Preferably, the plant extract agglomerates consist of extracts of more than one plant.

Preferably, the one or more plant extracts are organically derived.

According to a further aspect of the invention there is provided a beverage product comprising the above plant extract agglomerates.

Preferably the beverage product is a tea or herbal tea product.

Further preferably, the beverage product is packaged in a coffee pod or tea bag.

Throughout this specification and the claims which follow, unless the context requires otherwise, the word “comprise” and variations thereof such as “comprises” and “comprising”, will be understood to include the inclusion of a stated integer or step or group of integers or steps but not the exclusion of any other integer or step or groups of integers or steps.

The present summary is provided only by way of example, and not limitation. Other aspects of the present invention will be appreciated in view of the entirety of the present disclosure, including the entire text and claims.

### DETAILED DESCRIPTION

In their search to provide an alternative source, the inventors identified tea and similar beverages to be a suitable medium to introduce vitamins into modern diets. Tea and coffee are commonly drunk in many countries. Tea in particular is the most widely consumed beverage in the world and is used culturally as part of tea ceremonies, afternoon teas and the traditional greeting of visitors.

In addition to its taste, tea is commonly drunk for other benefits including increasing alertness as a result of its caffeine content. Tea also contains antioxidants such as polyphenols which have been considered to provide anticarcinogenic and antiarteriosclerotic effects. While tea has particular health benefits as identified above, it contains relatively few vitamins such as vitamins A, B6, D12, C, D, E, K, niacin, or riboflavin.

The inventors identified tea and similar beverage products including herbal teas (such as chrysanthemum, mint, and chamomile) and coffee as a suitable medium to introduce greater vitamin intake into modern diets, since they are already commonly drunk but contain relatively few vitamins. In this regard, the inventors have sought to develop a vitamin-rich product which:
(a) is suitable for mixing with various tea and other beverage compositions, at various and customisable rates of addition;
(b) does not segregate following mixing with teas and other dry beverage compositions;
(c) is readily dissolvable in hot water; and
(d) can be processed with typical beverage delivery means, such as processing with a tea bagging technologies.

As a first step, the inventors identified plant extract powder as a suitable source of vitamins, since the plant extract powders are naturally derived, optionally organic, and with suitable selection are able to cover a broad of daily vitamin needs. Suitable plant extracts include extracts of:
(a) agati (Sesbania grandiflora) which is believed to be a source of vitamins C, B9, B1 and B2, as well as iron and selenium;
(b) holy basil (Ocimum sanctum) which is believed to be a source of vitamins A, K, C, and B3 as well as magnesium, iron, potassium and calcium;
(c) lemon (Citrus limon) which is believed to be a source of vitamin C and B-complex vitamins, as well as calcium, copper, iron, magnesium, phosphorous and potassium;
(d) Indian gooseberry (Phyllanthus emblica), which is believed to be a source of B-complex vitamins as well as carotene, calcium, phosphorous and iron;
(e) common guava (Psidium guahava), which is believed to be a source of vitamins A, C, B-6, niacin, folate, thiamine and riboflavin; and
(f) achiote (Bixa orellana), which is believed to be a source of carotenoids, folate and calcium.

In addition, other suitable plant extracts include: chirchita (Achyranthes aspera) for saponins; pomegranate (Punica granatum) for polyphenols; pala indigo (Wrightia tinctoria) for calcium; mango (Mangifera indica) or amla (Emblica officinalis) for vitamin C; curry leaves (Murraya koenigii) for iron; Moringa (Moringa oleifera) for chromium; wild-sage (Lantana camara) for magnesium; mustard seeds (of the family Bassicaceae) for selenium; and bamboo shoots (Bambusa arundinacea) for silica;

It is believed that a mixture of the above plant extracts will provide a balanced mixture of vitamins suitable for use as a vitamin supplement product, noting that other plant extracts may be alternatively or additionally used.

While plant extracts were considered a suitable source of naturally derived nutrients, the inventors discovered that use of plant extract powders has its disadvantages. In particular, the inventors found that tea bag processing equipment, as conventionally used, can only process tea mixtures containing up to 5% plant extract powder content. This set an unsatisfactorily low limit on the amount of plant extract powder, and thereby nutrients, that could be included in a tea bag. In addition, the inventors identified the possibility that, due to density and size distribution differences, plant extract powders may segregate during transport and storage when mixed with beverage products such as loose leaved tea, thereby providing an uneven distribution of plant extract powder (and therefore nutrients) in the eventual beverage product.

Upon identification of the above issues, the inventors then considered and/or trialled several alternatives as now discussed.

The inventors initially considered coating the beverage product, e.g. tea leaves, with plant the extract powders (by spraying with a solution of the extract powders followed by drying and bind residual dry ingredients to the tea leaves) since coating of the beverage product could overcome segregation and powder processing issues during tea bagging. However, this was deemed a less than perfect solution since the resulting product would have limited processing flexibility. That is, it would than prove difficult to customise particular beverage and plant extract powder compositions following coating of the tea leaves. Also, it was identified that wetting and drying of the tea could negatively affect its fragrance and other properties.

The inventors thereafter trialled sintering or fusion style agglomeration and comminution of plant extract powders to provide an aggregated powder product. It was thought that agglomeration would overcome segregation and tea bag processing issues while suitably allowing for adaptability in beverage compositions. The considered fusion style agglomeration process was similar to that set out in U.S. Pat. No. 6,497,911. U.S. Pat. No. 6,497,911 B1, in the name of Hansen et al., discloses a method of forming a water soluble particulate material from an extract powder, e.g. tea or coffee extract. In the disclosed method a coffee or tea extract powder having a moisture content higher than that desired in the final product (e.g. 4-12%) is heat treated under compression at a sufficient temperature and for a sufficient time effect fusion of the powder into a cake-like structure. Following cooling the cake-like structure is comminuted and dried. However, the inventors identified that heating of the plant extract powders under such process conditions would have an unsatisfactorily detrimental effect on the ultimate nutrient content of the plant extract product. It was also identified that compression could have detrimentally affect water solubility as relevant to beverage products, since the pressure required to create a stable granule could lead to a hardening of the granule's exterior.

The inventors then explored a low temperature heat transfer drying process as provided under proprietary arrangements with a third party research company. It was hoped that by slowly heating the extract powders over time the temperature of the powder extracts could be suitably controlled to prevent over-heating and degrading nutrients. However, the applied process required use of an additive binder material. Since it was desired that the vitamin product contain minimal, if any, artificial additives the selection of suitable binders was limited and included materials such as apple or kale paste. These pastes altered the flavour of the tea while also becoming sticky when processed through tea bagging machinery. The resulting stickiness affected agglomerate flowability and ultimately prevented tea bag processing.

The inventors then explored agglomeration through rewet agglomeration. Under rewet agglomeration powders are wet and then redried under agitation. Wetting creates liquid bridges between powder particles. Upon re-drying the liquid bridges are converted to solid bridges, binding the powder particles together. Agitation during drying allows for control of the ultimate size of the agglomerates and improves the porosity of the granule, thereby improving its solubility.

Rewet agglomeration has previously been used to agglomerate food products such as milk powder and coffee powder as now discussed.

U.S. Pat. No. 4,490,403 A, in the name of Pisecky et al, discloses a method of drying and agglomerating milk powder. In the disclosed process milk liquid is atomised and sprayed into a central downward stream of drying gas at 200-400° C. to produce particles within a drying chamber. The lower part of the drying chamber contains a layer of particles fluidized in an upward stream of gas having a velocity of 0.3-1.5 m/s, whereby the temperature of the gas is adjusted to between 10-150° C. to ensure agglomeration. The stream of drying gas is regulated to ensure a moisture content in the fluidized layer of between 2-16%. The layer of fluidized particles is generally maintained at a temperature of 60-90° C. As inferable from U.S. Pat. No. 4,490,403 conditions in the fluidizing bed must be controlled to provide for suitable agglomeration. Low moisture content results in minimal agglomeration while high moisture content, insufficient temperatures, low flow-rates, etc. can lead to other issues such as excessive stickiness, insufficient drying and other matters which also negatively affect agglomeration.

U.S. Pat. No. 3,740,232 A, in the name of Purves et al, discloses a method of agglomerating instant coffee. According to the disclosed method a falling stream of coffee powder is intersected by a steam jets which wet and agglomerate the coffee powder and direct it onto a conveyor, The conveyor re-elevates the agglomerated coffee powder via a screen so that it can again fall and be intersected by steam. Fusion and agglomeration following the second steam treatment are carried out in a fluidized bed. Coffee agglomerates are held in the bed for a residence time of 30s and the temperature of air passing through the drier is about 250° F. (120° C.).

While rewet agglomeration was identified as a suitable means to agglomerate extract powders, the inventors through their previous research understood that use of high temperatures during processing could detrimentally affect the nutrient content of the resulting product. Trials were therefore held whereby extract powders were rewet agglomerated at low temperatures as now discussed.

### Trials

Batchwise trials were held using an Aeromatic AG Benchtop fluidised bed drier. The air inlet temperature was held 60° C. for each trial. Extract powders were inserted into the drier and pre-warmed by inlet air for a period of five minutes prior to spray application of water. Water was sprayed onto the extract powder for a period of several minutes, and fluidisation was thereafter continued to allow for drying of the agglomerates. Results of the trials were as follows:

TABLE 1

| | | | | | | Trial 6 (rework |
|---|---|---|---|---|---|---|
| | Trial 1 | Trial 2 | Trial 3 | Trial 4 | Trial 5 | material) |
| Initial Weight - powder (g) | 200 | 250 | 400 | 400 | 480 | 110 |
| Final Weight - agglomerate | 92 | 181.2 | 268 | 288.6 | 447.6 | 108.8 |
| product (g) | | | | | | |
| Yield (%) | 46.0 | 72.5 | 67.0 | 72.2 | 93.3 | 98.9 |
| Product Weight after sieving | 84.6 | 161.7 | 234.7 | 234.7 | 389.2 | 101.6 |
| (g) | | | | | | |
| Yield after sieving (%) | 42.3 | 64.7 | 58.7 | 58.7 | 81.1 | 92.4 |
| Air inlet temperature (° C.) | 60 | 60 | 60 | 60 | 60 | 60 |
| Pre-warming time before | 5 | 5 | 3 | 3 | 3 | 3 |
| water spray applied (min) | | | | | | |
| Total time of water spray - | 14 | 14 | 8.5 | 10 | 14 | 10 |
| including pauses (min) | | | | | | |
| Drying time - after water | 10 | 10 | 11 | 10 | 10 | 10 |
| spray (min) | | | | | | |
| Water dosing flow rate | 5 | 5 | 7 | 7 | 5 | 5 |
| (ml/min) | | | | | | |
| Water dosage before first | 35 | 40 | 55 | 50 | 40 | 40 |
| pause (ml) | | | | | | |
| Water dosage before second | 10 | 10 | 0 | 10 | 10 | 0 |
| pause (ml) | | | | | | |
| Water dosage before third | 0 | 5 | 0 | 10 | 10 | 0 |
| pause (ml) | | | | | | |
| Water dosage before fourth | 0 | 0 | 0 | 0 | 20 | 0 |
| pause (ml) | | | | | | |
| Total water dosage (ml) | 45 | 55 | 55 | 70 | 80 | 40 |
| Density of resulting | 484 | 420 | 450 | 370 | 450 | 460 |
| agglomerate (g/l) | | | | | | |

The results demonstrated that suitable agglomerates of plant extract powders could be obtained by fluidised bed rewet agglomeration at suitably low process temperatures. While agglomerate yield was lower than expected, it was concluded that this was due to a poor dust recovery mechanism in use with the trial drier. The density of the resulting agglomerates was found to be between 370-484 g/I which is similar to that of fannings grade leaf tea (450-500 g/I). By maintaining the inlet temperature at relatively low values (e.g. 60²C), the inventors were able to provide a plant extract agglomerate with minimal vitramin/nutrient loss.

With a suitable mixture of extract powders a single tea product may through entirely natural means provide a large portion of daily vitamin requirements though a single beverage. Plant extract granules, added at a rate of 0.411 g per tea bag (or 19% of tea bag contents, were able to provide the below results, obtained through independent testing performed on the invention.

TABLE 2

| | Average per 2 g tea bag | | | |
|---|---|---|---|---|
| | (200 ml boiling water) | | Average per 100 ml | |
| Energy | 1 | kCal (4 kJ) | 0.5 | kCal (2 kJ) |
| Fat, total | 0 | g | 0 | g |
| saturated | 0 | g | 0 | g |
| Carbohydrate | 0.2 | g | 0.1 | g |
| sugars, total | 0 | g | 0 | g |
| Protein | 0 | g | 0 | g |
| Salt (sodium) | 1.3 | mg | 0.6 | mg |
| Thiamin (B1) | 1.0 | mg (80% RDI) | 0.5 | mg |
| Riboflavin (B2) | 1.0 | mg (59% RDI) | 0.5 | mg |
| Niacin (B3) | 8.4 | mg (49% RDI) | 4.2 | mg |
| Pantothenic Acid (B5) | 2.4 | mg (48% RDI) | 1.2 | mg |
| Vitamin B6 | 2.0 | mg (100% RDI) | 1.0 | mg |
| Biotin (B7) | 100 | mcg (50% RDI) | 50 | mcg |
| Folate | 100 | mcg (48% RDI) | 50 | mcg |
| Vitamin C | 19 | mg (48% RDI) | 9.5 | mg |
| Zinc | 5.1 | mg (43% RDI) | 2.5 | mg |

Identified advantages capable of being achieved through the invention include:
(a) providing a natural source of vitamins and other nutrients;
(b) being readily suitable for incorporation into existing beverage products so that nutrients naturally form part of the existing modern diet;
(c) being readily dissolvable on hot water;
(d) being suitable for processing through existing tea bagging technologies at mixing rates of higher than 5%;
(e) providing an agglomerate density similar to loose leaf tea so as to avoid segregation in storage and transport;
(f) being adaptable so that the amount and composition of vitamins added to a particular beverage product can be varied;
(g) being suitable for those who have trouble ingesting tablets;
(h) providing minimal stomach irritation

Modifications and variations as would be deemed obvious to the person skilled in the art are included within the ambit of the present invention as claimed in the appended claims.

1. A method of producing plant extract agglomerates suitable for a beverage product, the method comprising rewet agglomerating plant extract powder wherein all processing and drying is performed at low temperatures to substantially avoid decreasing the nutritional value of the plant extract powder.

2. The method according to claim 1, wherein the method is performed using fluidized bed rewet agglomeration.

3. The method according to claim 1, wherein the processing and drying is performed at below 65° C.

4. The method according to claim 1, wherein the plant extract powder is wet with water.

5. The method according to claim 1, wherein the plant extract powder comprises one or more plant extracts of: agati (Sesbania grandiflora); holy basil (Ocimum sanctum), lemon (Citrus limon), Indian gooseberry (Phyllanthus emblica), common guava (Psidium guahava), and achiote (Bixa orellana).

6. A plant extract agglomerate produced according to the method of claim 1.

7. The plant extract agglomerate according to claim 6, wherein the plant extract agglomerate consists extracts of more than one plant.

8. The plant extract agglomerate according to claim either of claim 6, wherein the plant extract(s) are organically derived.

9. A beverage product comprising the plant extract agglomerate according to claim 6.

10. The beverage product according to claim 9, wherein the beverage product is a tea or herbal tea product.

11. The beverage product according to claim 9, wherein the beverage product is packaged in a coffee pod or a tea bag.

12. The method according to claim 1, wherein the method is performed using fluidized bed rewet agglomeration, wherein the plant extract powder is wet with water, and wherein the processing and drying are performed at below 65° C.

13. The method according to claim 12, wherein the plant extract powder comprises one or more plant extracts of: agati (Sesbania grandiflora); holy basil (Ocimum sanctum), lemon (Citrus limon), Indian gooseberry (Phyllanthus emblica), common guava (Psidium guahava), and achiote (Bixa orellana).
